(19) United States
(12) Patent Application Publication
(10) Pub. No.: US 2006/0106339 A1
(43) Pub. Date: May 18, 2006

(54) Medical device

(76) Inventor: Emmanuel Mastorakis, Fribourg (CH)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 10/523,293

(22) PCT Filed: Aug. 1, 2003
(86) PCT No.: PCT/EP03/08643

§ 371 Date: Sep. 8, 2005

(30) Foreign Application Priority Data
Aug. 6, 2002 (EP) 20020100365

(51) Int. Cl. A61M 5/00; A61M 5/31

(52) U.S. Cl. 604/110; 604/240

(57) ABSTRACT

A medical device such as a syringe (10) includes a retractable needle (20) and a tilting system (52). A tilting system (52) includes a deflector in the form of a ramp (83) for deflecting legs (66) of a needle retainer (18) of the device away from a longitudinal axis (90) thereof.

The present invention relates to a medical device, such as to a syringe, catheter or cannulas having a sharp device such as a needle. The medical device may be any medical device having a sharp device, such as a needle or knife. The invention relates, for example, to butterflies and may have application to infusion and transfusion sets and drips in which it is desirable to connect/disconnect to two or more medical devices, one including a sharp device such as a needle thereon.

To avoid undesirable needle stick injuries potentially involving the undesirable transfer of media through needles, attempts have been made to provide medical sharp devices such as safety syringes which are usable only once.

A known medical devise is disclosed in WO02/26295 A2, the content of which is hereby incorporated by reference. This known medical device has a medical sharp device, a retainer for retaining the sharp device and a moveable retractor adapted for connection with a connection member of the retainer and for moving the sharp device from a use position to a retracted position thereof, wherein a tilt system is provided for tilting the sharp device upon movement of the retainer to the retracted position. However, the tilting system, which employs asymmetrically configured lugs, requires close manufacturing tolerances and has been found to be more difficult than is desirable to manufacture for consistently reliable operation thereof.

In U.S. Pat. No. 5,104,378, a retraction probe is a taper fit in a so-called locking bore. The locking bore is sealed off by a plunger gasket front face having a so-called locating concavity. Due to the taper fit, which requires a low 2° taper angle, the probe is relatively long. This means the syringe barrel has to be excessively long for fluid drawback purposes since a substantial amount of air has to be expelled after fluid drawback. The closed front of the plunger gasket front face, it is believed, is intended to prevent oily or otherwise slippery fluids in the syringe from stopping the taper fit of probe into locking bore from working. The gasket front face has to be pierced by the probe during injection, possibly causing pain to the patient and air can be forced by the volume of the probe from behind the pierced gasket front face to the front side thereof whereby it may dangerously be injected into a patient.

In EP-A-0636381 a single lug is provided with a locking element, another lug operating as a guide causing a needle to be tilted as it is withdrawn inside syringe. The single locking element is considered to be likely to be weak and tilting may not be consistent.

In U.S. Pat. No. 5,328,484 a needle holder has four legs which have ends on four sides of a central axis of the syringe. One of the legs is engaged by a half-cone hook for tilting. The needle holder is complicated to make with the four legs that appear to be required to cause tilted whatever angle the needle is rotationally positioned in about the longitudinal axis of the syringe.

In U.S. Pat. No. 5,378,240 a single catch on a needle mount engages behind a radial flange. Similar problems to those of EP-A-0636381 are likely.

The present invention aims to provide an improved medical device and to address at least to a certain extent the problems of the prior art.

According to one aspect of the present invention, a medical device has the features as set out in claim 1. Another aspect is set out in claim 30. Various optional features are mentioned in the dependant claims.

The deflector on the retractor enables positive deflection of the retainer. The deflector, when deflecting the retainer is preferably spaced from a pivot region of the retainer along a longitudinal axis of the medical device. This spacing and the positive deflection force enable a reliable and effective torque to be provided for tilting purposes.

Preferably, the medical sharp device comprises a needle, such as a hypodermic needle,

The medical device may comprise a syringe, the retractor being formed on a plunger of the syringe. The syringe may have a cylindrical barrel, a shoulder formed at one end thereof and a cylindrical neck formed on the shoulder.

Preferably, the retainer is located in the neck and a hub is provided for preventing forward movement of the retainer out of the neck.

The medical device is preferably generally elongated in shape having a front end from which the medical sharp device in one configuration thereof extends forwards, and a rear end.

The retainer may include a medical sharp device retaining portion. The retaining portion may be cylindrical in shape. When the medical device comprises a syringe having a neck, the retaining portion is preferably cylindrical, fitting snugly inside the neck in one configuration thereof.

Preferably, the connection member comprises a flexible leg. More preferably, two said flexible legs are provided. The flexible legs are preferably formed in a diamond shape.

Preferably, each flexible leg is joined at one end to a sharp device retaining portion of the retainer and at the other distal end thereof to the other flexible leg.

Preferably, the distal ends form a curved apex. The curved apex may be adapted to engage the deflector during connection of the retractor to the retainer. The curved apex may be curved in two or more different directions. The curved apex may be domed. Curved surfaces are advantageous in this regard in that they enable the curved apex and legs to smoothly ride over the deflector.

Preferably, the joined distal end forms an apex which is located on a longitudinal axis of the medical device. The deflector is preferably positioned to engage the apex and thus move the apex away from the longitudinal axis of the device. Preferably the apex is curved.

Preferably, at least one said leg includes a stop part adapted to releasably abut against a retainer surface inside a main body of the device. The main body of the device may, in the case of a syringe, comprise a barrel of the syringe.

The stop part may comprise a ledge and the retainer surface may comprise an annular recess located in the main body.

Preferably, at least one leg includes a formation adapted to lock with the moveable retractor. The formation preferably comprises a ledge.

Preferably, the retractor has a catch for engaging the formation. The catch and/or ledge when provided allow a very positive locking of the at least one leg and the retractor.

Preferably, the retractor has an entrance having an entrance ledge to engage the formation.

Preferably, the deflector comprises an abutment, preferably for guiding the leg away from a longitudinal axis of the device.

Preferably, the abutment comprises a ramp. The ramp is preferably spaced along a longitudinal axis of the medical device from the catch. This enables effective and reliable deflection of the connection member for tilting purposes.

Preferably, the ramp is inclined at an angle of about 70 degrees to a longitudinal axis of the medical device. The ramp may, for example, be inclined at between 30 and 80 degrees to a longitudinal axis, 50 to 70 degrees being preferred.

Preferably, the ramp is formed in an interior space of the retractor, and the ramp is preferably spaced from a rear surface of the interior space by a wall which is substantially parallel to the longitudinal axis of the device. Preferably, the wall is aligned with the longitudinal axis. Thus, the apex formed by the joined distal ends of the legs may engage the ramp and then be deflected to one side for tilting purposes, with further movement of the apex into the interior space being towards the rear surface thereof.

Preferably, the features of claim 19 are provided. The catch and ledge may provide therefore a very reliable locking action.

Preferably, the features of claim 20 are provided. Thus the plunger may before locking of the retractor to the connection member be pushed very close to the front of the syringe barrel therefore allowing minimal syringe drawback before expelling air with further forwards pressure on the retractor causing locking to occur.

Preferably the features of claim 23 are provided. This allows insertion of the leg with the retractor and deflection of the leg without substantial frictional forces.

The present invention may be carried out in various ways and a preferred embodiment of a medical device in accordance with the present invention will now be described by way of example with reference to the accompanying drawings, in which:

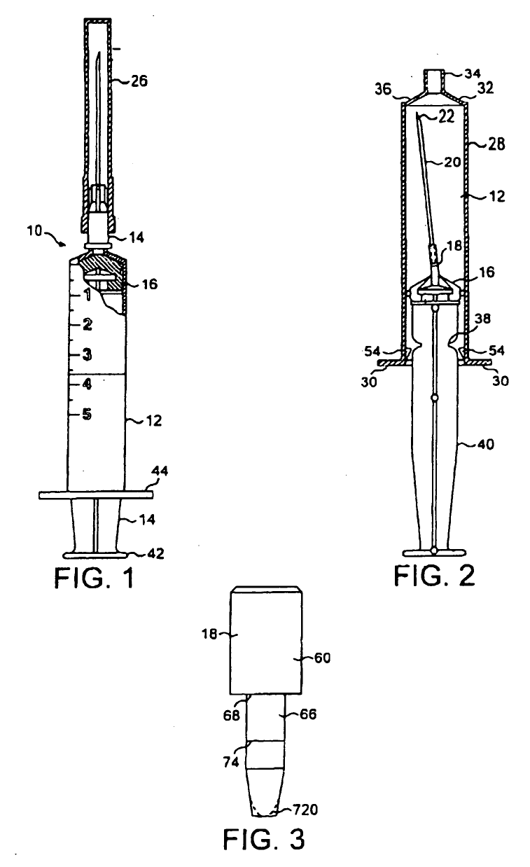

FIG. 1 is a partially cut away elevation of a medical device in the form of a syringe in accordance with a preferred embodiment of the present invention;

FIG. 2 typically shows the syringe of FIG. 1, after use, with a needle thereon in a tilted configuration;

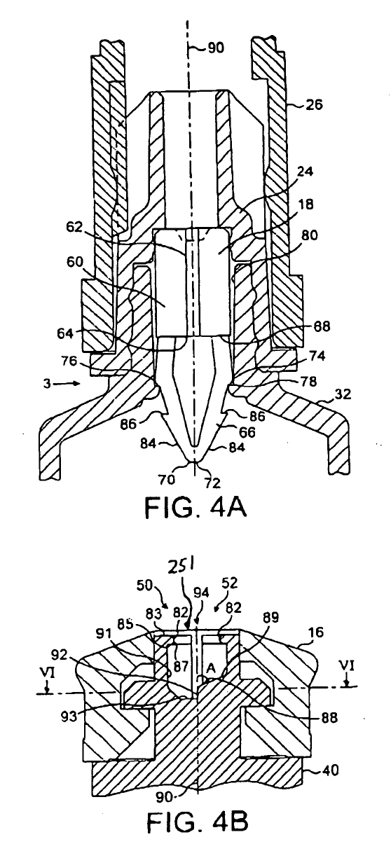

FIG. 3 is an elevation of the needle retainer in the direction 3 of FIG. 4a;

FIG. 4a is a partial cross-section of a barrel, needle retainer, hub and sheath of the syringe of FIG. 1;

FIG. 4b is a partial cross-section of a plunger stem and piston of the syringe of FIG. 1;

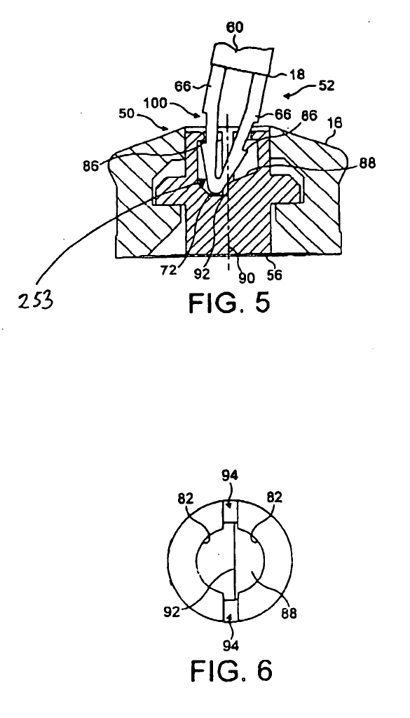

FIG. 5 corresponds to part of FIG. 4b, showing engagement between legs of the needle retainer and the retractor, with the needle retainer in a tilted configuration; and

FIG. 6 is a schematic cross-section on the plane VI-VI in FIG. 4b;

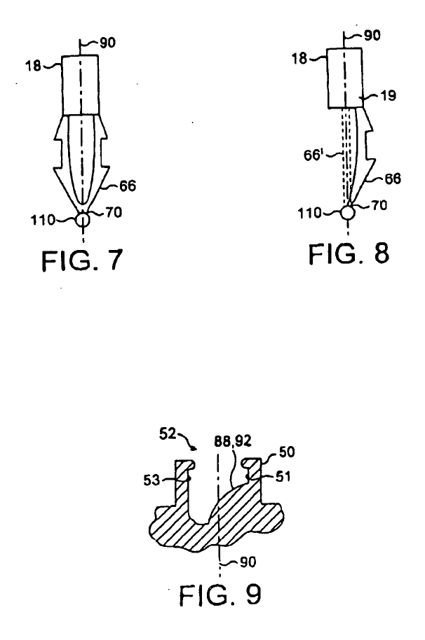

FIG. 7 shows a modification to the retainer (18) of FIG. 4a;

FIG. 8 shows a further modification of the retainer (18), having only one leg (66); and

FIG. 9 shows a modification to the deflector to that shown in FIG. 4b, in that a curved ramp/wall are provided.

As shown in FIGS. 1 and 2, a preferred embodiment of a medical device in the form of a syringe (10) in accordance with the present invention comprises a cylindrical barrel (12), a plunger (14), a piston (16), a needle retainer (18), a hypodermic needle (20) with a sharp tip end (22), a hub (24) and a needle sheath (26).

As shown in FIGS. 1 and 2, the barrel (12) has a main cylindrical part (28) having finger tabs (30) located at a rear end thereof and a shoulder (32) and small diameter cylindrical neck (34) formed at a front end (36) thereof. Although the piston (16) and plunger stem (40) are shown somewhat schematically in FIG. 2, it will be appreciated that the plunger stem (40) includes frangible weakened portions (38) to assist with snapping of the stem (40) after use, to assist in preventing the use of the syringe (10).

In use, the syringe (10) may be presented to the user with the piston (16) spaced rearwardly of the front end (36) of the barrel cylindrical part (28). The sheath (26) may be removed and the piston (16) may be moved using the knob (42) towards the rear end (44) of the cylindrical main part (28) of the barrel (12) in order to draw fluid (not shown) into the barrel (12). Air (not shown) may then be expelled from the syringe (10) in accordance with standard procedure and the syringe may then be used for injection purposes. Once the piston (16) reaches the front end (36) of the main cylindrical part (28) of the barrel (12), a retractor (50) (see FIG. 4b) formed on the front of the stem (40) engages the needle retainer (18) and the retractor (50) may then be pulled rearwardly again using the knob (42) until the tip (22) of the needle (20) passes the neck (32) and the needle (20) is tilted by a tilting system (52), formed by the retractor (50) and needle retainer (18) (see FIGS. 2 and 5). Once tilted, as shown in FIG. 2, the needle cannot be pushed forwards again through the neck (34). Additionally, the barrel includes stops (54) for preventing the piston (16) from being removed rearwardly from the barrel. Thus, once used and in the tilled configuration, the needle (20) cannot be reused. Therefore, needle stick injuries are desirably prevented from occurring and, furthermore, the possibility of reuse of the syringe (10) is minimised, such that the needle (20) is not used for procedures on more than one human or animal and the risk of the transfer of undesirable materials between the same is minimised.

It will be appreciated that, as well as being useable for injection purposes, the syringe (10) may be used for withdrawing samples, such as blood or other fluids from humans or animals or for other purposes.

With reference to FIG. 4a and FIG. 3, the needle retainer (18) includes a cylindrical portion (60) which sits snugly inside the neck (34). The cylindrical portion includes a through-bore (62) into which the needle (20) is a press fit to a position in which a rear end (not shown) of the needle (20) is located at a rear end (64) of the through-bore (62). In other embodiments, the rear end of the needle may be located in front of or to the rear of the rear end (64) of the bore (62) as described. Two flexible legs (66) are attached at front ends (68) thereof to the cylindrical portion (60) of the needle retainer (18). As shown in FIG. 4a, the flexible legs (66) form a diamond shape and are joined at distal ends (70) thereof to form a curved apex (72), which is domed. Each leg (66) includes a flat ledge (74) which is in the use configuration of FIG. 4a engaged with an annular ledge (76) at a rear end (78) of an internal bore (80) of neck (34).

As the plunger (16) is moved forwards, the annular ledges (82) of the retractor (50) engage against rear wedging surfaces (84) of the legs (66), resiliently wedging and pushing the legs (66) together so that the ledges (74) disengage from the annular ledge (76). Furthermore, ledges (86) on the legs (66) engage behind annular ledges (82) of the retractor (50) and the curved apex (72) engages an internal ramp (88) of the retractor (50) and is resiliently pushed to one side of the longitudinal axis (90) of the syringe (10) and is then prevented from returning to the longitudinal axis (90) by a substantially vertical wall (92) of the retractor (50). The legs (66) are therefore essentially bent to one side by the retractor in the example, with the cylindrical portion (60) remaining in line with the axis (90) to the left of the position shown in FIG. 4a by the retractor (50). As the needle tip (22) passes the neck (34) and shoulder 32, the legs (66) adopts the configuration shown in FIG. 2. The cylindrical portion (60) of the needle retainer (18) is tilted (sec FIG. 5) with respect to the longitudinal axis of the syringe (10), with at least one of the ledges (86) engaged with at least one of the ledges (82), and with the apex (72) formed by the joined distal ends of the legs (66) located to the side of the substantially vertical wall (92), by which the apex (72) is retained away from the axis (90).

FIG. 5 shows only one engaged configuration of the legs (66) once retained by the retractor (50). If the legs (66) engage the retractor (50) when in another location or configuration, such as when the legs are located in a position rotated at 90 degrees or any other angle around the longitudinal axis from that shown in FIGS. 4a and 5, such that the engagement with the ramp (88) is different, the apex and legs will still be deflected by the ramp (88) and then held to the side of the axis (90) by the wall (92) due to the configuration of the legs (66) (with their apex initially on the central axis (90)), ramp (88) and wall (92). The apex is preferably curved in two directions as shown by dotted line 720 as a modification in FIG. 3 (the curve in the other direction being as in FIG. 4a. Thus, the apex is preferably domed.

The ramp (88) is inclined at an angle A (see FIG. 4b) to the longitudinal axis (90) of the syringe (10) of 70″. The ramp is generally flat but may be curved. The wall (92) is also generally flat but may be curved in some other embodiments.

It will be seen that the ledges (82) of the retractor (50) are spaced apart by gaps (94). These gaps (94) provide some resilience for the retractor (50) to facilitate the engagement with the legs (66).

The retractor (50) may be formed integrally with or separately from the stem (40). Preferably, these parts are formed integrally. Thus, it will be seen that the syringe (10) may have a particularly simple construction consisting of only seven parts to be assembled together, namely the sheath (26), hub (24), needle retainer (18), needle (20), barrel (12), stem (40)/retractor (50) and piston (16).

The deflector of the tilting system (52) formed by the configuration of the abutment ramp (88), wall (92) and legs (66) has been found to be particularly reliable and effective in practice. The use of the two flexible legs joined at the distal ends thereof provides a firm engagement with the interior ledge (76) of the neck (34). Despite this effective resilience, the legs (66) may be reliably squeezed together by the retractor (50) for disengagement from the ledge (76) and the needle (20) may then be reliably retracted into the syringe (10) and tilted in order to minimise the possibility of reuse. The ramp (88) and wall (92) are spaced longitudinally from a pivot region (100) of the retainer (18) and/or of retractor, namely from the region of the formations (86,74) and/or the entrance ledges (82) of the retractor, and this spaced configuration allows a reliable torque to be applied to the retainer (18) for tilting.

FIG. 7 shows a modification to the needle retainer (18) in which two legs (66) have distal ends (70) having a curved formation or ball (110) formed thereon. The end (70) of the legs (66) and the ball (110) are located on the central axis (90) of the needle retainer (18) and medical device. The central nature of the ball (110) and the way in which the romp/wall (88/92), extend across the axis (90) ensure that the needle (20) is tilted reliably.

FIG. 8 shows a modification in which the needle retainer (18) only has one leg (66) having a distal end (70) which has a curved formation or ball like element (110) located thereon, the curved element or ball being located on the central axis (90) of the needle retainer (18) upper portion (19) and/or of the medical device. Although only one leg (66) is present, the location of the end (70) thereof on the central axis (90) and the configuration of the ramp (88) and wall (92) ensure that the tilting of the needle (20) is reliable.

As shown in FIG. 9, the abutment may comprise a curved ramp/wall (88/92) of increasing steepness across the a retractor (50) from one side (51) to another side (53) thereof. As in the embodiments shown in FIG. 4b, the ramp/wall (88/92) extend across to the longitudinal axis (90) or slightly further than the central axis from the side (51).

In the embodiment of FIG. 4b, the top (89) of the ramp (88) is located at a distance 1.1 mm above 2 lower surface (93) of the interior of the retractor (50). The distance from the lower surface (93) to upper surfaces (83) of ledges (82) is 3 mm. The distance between upper surfaces (83) of the ledges (82) and lower surfaces (85) of the ledges (82) is 0.5 mm. An interior cross dimension or diameter between facing surfaces (87) of the ledges (82) is 1.7 mm and an interior cross dimension or diameter of the retractor (50) between opposing interior side walls (91) of the retractor is about 2-2.3 mm. preferably, the height of the deflector to the point (89) from a lower surface (93) of the inside of the retractor (50) is about 30%-40% of the height of the interior of the retractor from the lower surface (93) to upper surfaces (83) thereof. This height of the deflector may be about 40%-60% of a cross dimension or diameter of the retractor between interior side walls (91) thereof. The ramp (88) and wall (92) may be flat, as in FIG. 4b. Alternatively, it may be curved in one or two directions. A curved ramp/wall (88/92) are shown in FIG. 9, this deflector being curved in only one dimension and extending consistently across the retractor as shown by the nature of the cross section in FIG. 9.

The deflector/leg configurations in the various embodiments allow the tilting of the needle (20) to work reliably in any rotational configuration of the needle (20) and retainer (18) about the longitudinal axis (90) relative to the deflector (88, 92). In particular, this reliability is aided by the way in which the deflector extends to or slightly across the central axis (90) and in that distal end (70)/domed ends (72,110) of leg(s) are located on or near to the central axis (90) in the use configuration of the needle (20). The embodiment of FIG. 8 may be farther modified by replacing the off centre leg (66) with a single central leg (66)—having the curved end (110) as shown in FIG. 8 in dashed lines.

Furthermore, before the ledges 86 have engaged the retractor catch, the retractor may be freely pushed up to engage the surfaces 84 without locking, so that liquid or other material can then be freely drawn back with a minimum further air expulsion step then being needed. This is possible since the legs can enter locking cavity 251 of retractor freely without locking thereto and without disengaging from ledge (76). Only upon fill insertion of the legs 70,72 do the legs lock to the retractor.

As shown in FIG. 5, the legs 70,72, or at least the distal ends thereof are deflected by ramp 88 into open side cavity 253 of locking chamber 251, the side cavity allowing this deflection with minimal friction forces.

Various modifications may be made to the specific embodiment described without departing from the scope of the invention as defined by the claims as interpreted under patent law.

1. A medical device having a medical sharp device, a retainer for retaining the sharp device and a movable retractor adopted for connection with a connection member of the retainer and for moving the sharp device from a use position to a more retracted position thereof, wherein a tilt system is provided for tilting the sharp device upon movement of the sharp device to the more retracted position thereof, the tilt system including a deflector on the retractor for deflecting the connection member during movement of the retractor into engagement with the retainer, the connection member having an engagement part thereof on a longitudinal axis, the engagement part being deflected off the axis by the deflector.

2. A medical device as claimed in claim 1 in which the medical sharp device comprises a needle.

3. A medical device as claimed in claim 1 which comprises a syringe, the retractor being formed on a plunger of the syringe.

4. A medical device as claimed in claim 3 in which the syringe has a cylindrical barrel, a shoulder formed at one end thereof and a cylindrical neck formed on the shoulder.

5. A medical device as claimed in claim 4 in which the retainer is located in the neck, and in which a hub is provided for preventing forward movement of the retainer out of the neck.

6. A medical device as claimed in claim 1 in which the retainer includes a cylindrical sharp device retaining portion.

7. A medical device as claimed in claim 1 in which the connection member has one end connected to a sharp retaining portion of the retainer and another, distal end thereof located on a longitudinal axis of the medical device in one configuration thereof.

8. A medical device as claimed in claim 7 in which the distal end of the connection member engages the deflector.

9. A medical device as claimed in claim 1 in which the connection member comprises a flexible leg.

10. A medical device as claimed in claim 9 in which two said flexible legs are provided, preferably formed in a diamond shape.

11. A medical device as claimed in claim 10 in which each flexible leg is joined at one end thereof to a sharp retaining portion of the retainer and at the other, distal end thereof to the other flexible leg.

12. A medical device as claimed in claim 7 in which a curved (preferably domed) nose is formed on the distal end of at least one connection member.

13. A medical device as claimed in claim 11 in which the joined distal ends form an apex which is located on a longitudinal axis of the medical device in one configuration thereof.

14. A medical device as claimed in claim 13 in which the apex is domed.

15. A medical device as claimed in claim 9 in which at least one leg includes a stop part adapted to releasably abut against a retainer surface inside a main body of the medical device.

16. A medical device as claimed in claim 15 in which the stop part comprises a ledge and in which the retainer surface comprises an annular recess located in the main body.

17. A medical device as claimed in claim 9 in which at least one leg includes a formation adapted to lock with the moveable retractor.

18. A medical device as claimed in claim 17 in which the formation comprises a ledge.

19. A medical device as claimed in claim 18 in which the retractor has a catch for positively locking with the ledge, preferably with generally planar abutment faces of the catch and ledge abutting one another.

20. A medical device as claimed in claim 17 in which the retractor has a locking cavity and the at least one leg is freely insertable and removable from said cavity in intermediate positions of the leg relative to the cavity and in which the leg and cavity are configured such that the locking of the leg to the retractor occurs upon a full insertion of the leg into the cavity.

21. A medical device as claimed in claim 17 in which the retractor has an entrance having an entrance ledge adapted to engage the formation.

22. A medical device as claimed in claim 9 in which the deflector comprises an abutment for guiding the leg away from a longitudinal axis of the medical device.

23. A device as in claim 22 in which the abutment is adapted to guide the leg sideways into a side chamber portion of the retractor.

24. A medical device as claimed in claim 22 in which the abutment includes a ramp.

25. A medical device as claimed in claim 24 in which the ramp is inclined at an angle of about 70 degrees to a longitudinal axis of the medical device.

26. A medical device as claimed in claim 24 in which the ramp is formed in an interior space of the retractor, and in which the ramp is spaced from a rear surface of the interior spaced by a wall substantially parallel to a longitudinal axis of the device.

27. A medical device as claimed in claim 26 in which the wall is aligned with the longitudinal axis of the medical device.

28. A medical device as claimed in claim 22 in which the abutment extends across the longitudinal axis.

29. A medical device as claimed in claim 28 in which at least part of the wall is located on an opposite side of the longitudinal axis to at least part of the ramp.

30. A medical device having a medical sharp device, a retainer for retaining the sharp device and a movable retractor adapted for connection with a connection member of the retainer and for moving the sharp device from a use position to a more retracted position thereof, wherein a tilt system is provided for tilting the sharp device upon movement of the sharp device to the more retracted position thereof, the tilt system including a deflector on the retractor for deflecting the connection member, the connection member having a portion thereof on a central axis of the retainer which engages the deflector and is guided away from the central axis by the deflector.
